(19) United States
(12) Patent Application Publication
(10) Pub. No.: US 2024/0390870 A1
(43) Pub. Date: Nov. 28, 2024

(54) APPARATUS FOR MANUFACTURING CUSTOMIZED COSMETIC PRODUCTS

(71) Applicant: LILLYCOVER, INC., Daegu (KR)

(72) Inventor: Sun Hee AN, Gyeongsan-si (KR)

(21) Appl. No.: 18/669,530

(22) Filed: May 21, 2024

(30) Foreign Application Priority Data
May 26, 2023 (KR) 10-2023-0068681

(51) Int. Cl. B01F 29/10; A45D 40/00; B01F 35/222; B01F 101/21

(52) U.S. Cl. CPC B01F 29/10; B01F 35/222; A45D 40/00; B01F 2101/21

(57) ABSTRACT

An apparatus for manufacturing customized cosmetic products includes a container injector configured to inject a container, a robot arm configured to move the injected container to a location needed for each process, a raw material injector configured to inject raw materials for a product into the container, a raw material supply unit configured to supply the raw materials to the raw material injector, and a stirring unit configured to mix the raw materials injected into the container, wherein the raw material supply unit includes a mounting panel on which a raw material pack is mounted and a detection panel arranged on one surface of the mounting panel, and the mounting panel has elasticity and is in contact with or detached from the detection panel according to a remaining amount of the raw material pack.

### CROSS-REFERENCE TO RELATED APPLICATION

This application claims priority from and the benefit of Korean Patent Application No. 10-2023-0068681, filed on May 26, 2023, which is hereby incorporated by reference for all purposes as if fully set forth herein.

### BACKGROUND

### Field

The disclosure relates to an apparatus for manufacturing customized cosmetic products.

### Discussion of the Background

Cosmetic products are used to add charm, brighten appearance, or maintain or promote skin health by cleaning and beautifying the human body.

Recently, cosmetic products that may be used in various ways according to skin conditions of people have been released. However, the skin conditions of the people may be different due to various causes such as age, skin trouble, and genetic factors. Accordingly, even though various types of cosmetic products are being released, all of various skin conditions of people may not be easily satisfied. Therefore, there is a need to manufacture customized cosmetic products in which components and contents of materials are determined according to a skin condition of a user.

### SUMMARY

Embodiments provide an apparatus for automatically manufacturing customized cosmetic products by selecting a material appropriate for the skin of a user.

Additional aspects will be set forth in part in the description which follows and, in part, will be apparent from the description, or may be learned by practice of the presented embodiments of the disclosure.

According to an embodiment, an apparatus for manufacturing customized cosmetic products includes a container injector configured to inject a container, a robot arm configured to move the injected container to a location needed for each process, a raw material injector configured to inject raw materials for a product into the container, a raw material supply unit configured to supply the raw materials to the raw material injector, and a stirring unit configured to mix the raw materials injected into the container, wherein the raw material supply unit includes a mounting panel on which a raw material pack is mounted and a detection panel arranged on one surface of the mounting panel, and the mounting panel has elasticity and is in contact with or detached from the detection panel according to a remaining amount of the raw material pack.

The detection panel may include a plurality of detection units formed to be spaced apart from one another along one surface of the detection panel, a signal may be generated by contact or separation between the mounting panel and the detection units, and the raw material pack, which needs to be replaced, may be determined according to the signal.

The mounting panel may include a body portion formed in a straight line and a plurality of protrusion portions spaced apart from one another on one side of the body portion and formed perpendicular to the body portion, raw material packs may be mounted on the protrusion portions, and the protrusion portions may be in contact with or detached from the detection panel according to remaining amounts of the raw material packs.

The apparatus may further include an inspector configured to distinguish between containers having different capacities, wherein the inspector may include a lever seesawing around a central axis, a pin arranged on one side of the lever and movable in a vertical direction, and when the pin rises, the one side of the lever may be raised by the pin, the other side of the lever may be lowered around the central axis, and the first sensor unit may detect a location of the other side of the lever.

The first sensor unit may include a plurality of sensors arranged in the vertical direction, and the sensors may detect the location of the other side of the lever, and the containers having different capacities may be distinguished by the location of the other side of the lever.

The pin may be raised by the container, and the container may be arranged perpendicular to the pin by the robot arm and is pushed up by the robot arm.

The stirring unit may include a seating unit on which the container is seated, the seating unit may rotate in a first direction, and the container may rotate in a second direction within the seating unit.

The apparatus may further include a second sensor unit including a pair of sensors arranged symmetrically around the stirring unit.

The pair of sensors may include photo sensors, and the container may be located between the pair of sensors.

The apparatus may further include a first discharge unit and a second discharge unit configured to discharge the container, wherein the first discharge unit accommodates the container in which the raw materials are mixed by the stirring unit, and the second discharge unit accommodates the container determined to be inappropriate by the inspector.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a perspective view schematically illustrating an apparatus for manufacturing customized cosmetic products, according to an embodiment;

FIG. 2 is a perspective view schematically illustrating a portion of the apparatus of FIG. 1 for manufacturing customized cosmetic products;

FIG. 3 is a plan view schematically illustrating a raw material injector of the apparatus of FIG. 1 for manufacturing customized cosmetic products;

FIG. 4A is a plan view schematically illustrating a raw material supply unit of the apparatus of FIG. 1 for manufacturing customized cosmetic products;

FIG. 4B is an exploded plan view of the raw material supply unit of FIG. 4A when viewed from a direction A;

FIG. 4C is a plan view of the raw material supply unit of FIG. 4A when viewed from a direction B;

FIG. 5 is a perspective view schematically illustrating a stirring unit of the apparatus of FIG. 1 for manufacturing customized cosmetic products;

FIG. 6A is a perspective view schematically illustrating an inspector of the apparatus of FIG. 2 for manufacturing customized cosmetic products; and

FIGS. 6B and 6C are plan views schematically illustrating an inspector of FIG. 6A of the apparatus for manufacturing customized cosmetic products.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

The disclosure may be modified in various ways and may have various embodiments, and thus, particular embodiments will be illustrated in the drawings and described in detail in the description. Advantages and features of the disclosure, and methods of achieving the same will become clear with reference to the description of embodiments taken in conjunction with the accompanying drawings. However, the disclosure is not limited to embodiments presented below, but may be implemented in various different forms.

In the following embodiments, the terms first, second, etc. may be only used herein to distinguish one component from another, not for limited sense.

In the following embodiments, the singular forms are intended to include the plural forms as well, unless the context clearly indicates otherwise.

The terms “comprise”, “include”, and/or “have” when used herein, specify the presence of stated features, elements, and/or components, but do not preclude the presence or addition of one or more other features, elements, and/or components.

In the following embodiment, when a portion, such as a layer, a region, or a component, is referred to as being “on,” or “above” another portion, it may be directly on the other portion or an intervening layer, region, or component may be present.

In the drawings, the sizes of components may be exaggerated or reduced for convenience of description. For example, the size and thickness of each component shown in the drawings are shown arbitrarily for convenience of description, and thus, the disclosure is not necessarily limited to those shown.

Hereinafter, embodiments will be described in detail with reference to the accompanying drawings, and when described with reference to the drawings, identical or corresponding components will be assigned the same reference numerals.

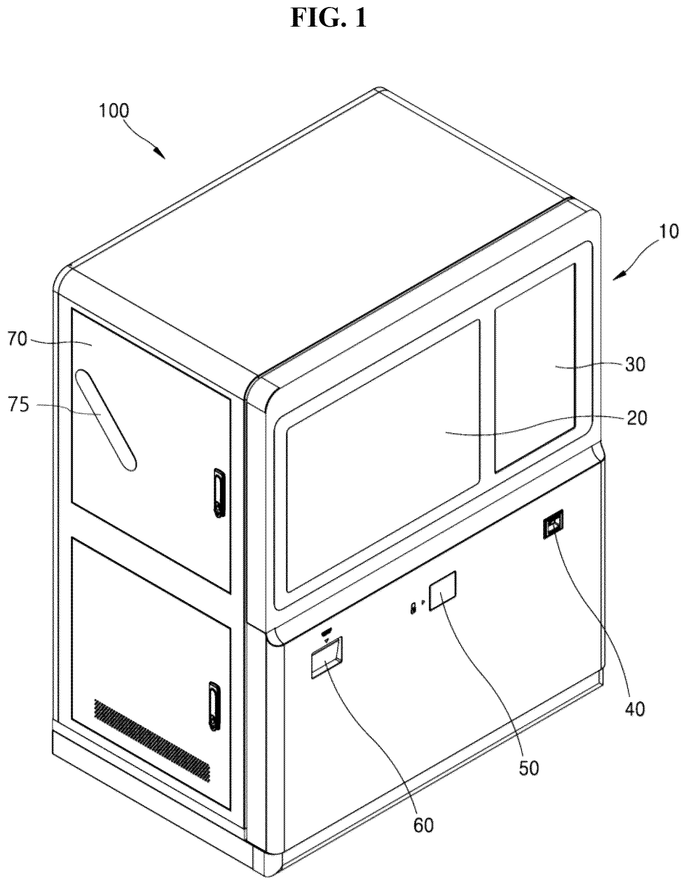

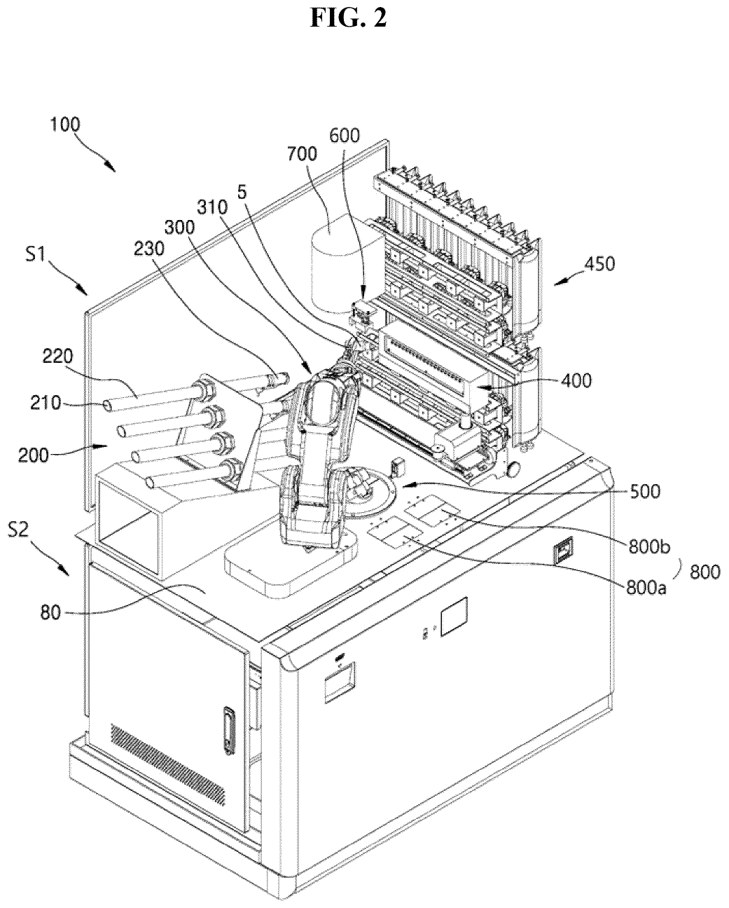

FIG. 1 is a perspective view schematically illustrating an apparatus for manufacturing customized cosmetic products, according to an embodiment, and FIG. 2 is a perspective view schematically illustrating a portion of the apparatus of FIG. 1 for manufacturing customized cosmetic products.

Referring to FIGS. 1 and 2, an apparatus 100 for manufacturing customized cosmetic products (hereinafter, referred to as a manufacturing apparatus) may include a housing 10, a transparent window 20 formed in the housing 10, a display unit 30, a barcode scanner 40, an outlet 50, a label output unit 60, a door 70, an upper plate 80, a container injector 200, a robot arm 300, a raw material injector 400, a stirring unit 500, a stopper processing unit 700, and a discharge unit 800.

The housing 10 may form the external appearance of the apparatus 100 for manufacturing customized cosmetic products. As an example, the housing 10 may have a hexahedral shape, and four pillars (not shown) may be located at vertices of the bottom of the hexahedral shape. The four pillars (not shown) may prevent the manufacturing apparatus 100 from tilting in any direction by adjusting the horizontality of the manufacturing apparatus 100. In addition, the housing 10 may include, on one surface thereof, the transparent window 20, the display unit 30, the barcode scanner 40, the outlet 50, and the label output unit 60. In addition, the housing 10 may include a plurality of doors 70 on remaining surfaces excluding one surface, an upper surface, and a lower surface thereof and may manage the interior of the manufacturing apparatus 100 through the doors 70.

The transparent window 20 may be formed to be transparent so that a manufacturing process of the manufacturing apparatus 100 may be identified in real time from the outside. The transparent window 20 may include any material that may be formed to be transparent and may include, for example, a transparent polymer compound or glass.

The display unit 30 may provide a user with information needed for manufacturing cosmetic products. As an example, the user may order a desired cosmetic product and proceed with payment, through the display unit 30.

The barcode scanner 40 may scan a barcode or QR code containing recipe information regarding a cosmetic product, so that an order may be generated without the user needing to input materials and contents of the cosmetic product one by one.

The outlet 50 may operate as a passage connecting the inside and outside of the manufacturing apparatus 100 to allow a cosmetic product completed inside the manufacturing apparatus 100 to be discharged to the outside.

The label output unit 60 may output a sticker including information regarding the completed cosmetic product, a logo of the product, and the like.

Meanwhile, the interior of the housing 10 may be divided into an upper space S1 and a lower space S2 by the upper plate 80.

The upper plate 80 may form a bottom surface of the upper space S1 and may simultaneously form an upper surface of the lower space S2. Also, a lower plate (not shown) may be formed on a bottom surface of the lower space S2.

A power distribution box (not shown) for electrical connections between components of the manufacturing apparatus 100, a controller (not shown) for controlling an operation of the manufacturing apparatus 100, a container storage unit (not shown) for storing a container 5 of the cosmetic product, a raw material storage unit (not shown) for storing cosmetic raw materials, a label issuer (not shown), and the like may be located on the lower plate (not shown).

Components for manufacturing the cosmetic product may be disposed on the upper plate 80. In detail, the upper plate 80 may have disposed thereon the container injector 200 into which the container 5 of the cosmetic product is injected, the robot arm 300 that moves the injected container 5 to a needed location for each process, the raw material injector 400 that injects raw materials into the container 5, the raw material supply unit 450 that supplies the raw materials to the raw material injector 400, and the stirring unit 500 that mixes the raw materials injected into the container 5.

The container injector 200 may include a container injection inlet 210, a sliding unit 220, and an arrival unit 230. As an example, the container injection inlet 210 of the container injector 200 may be covered by an auxiliary door 75. The container injection inlet 210 may be covered by the auxiliary door 75, and thus, when the container 5 is injected, the entire door 70 does not need to be opened and foreign substances may be prevented from entering the container injector 200. Meanwhile, the container 5 may include two containers 5 having different capacities, and the two containers 5 having different capacities may have, for example, the same diameter and different lengths. The two containers 5 having different capacities may be separately injected into different container injection inlets 210. When the container 5 is injected into the container injection inlet 210, the container 5 may move along the sliding unit 220 connecting the container injection inlet 210 to the arrival unit 230. Here, the container injection inlet 210 may be located higher than the arrival unit 230, and thus, the sliding unit 220 may have a slope, and the container 5 may be seated on the arrival unit 230 while sliding along the inclined sliding unit 220. In addition, a plurality of containers 5 may be loaded on the container injector 200 along the sliding unit 220. When the plurality of containers 5 are loaded along the sliding unit 220, the plurality of containers 5 may sequentially slide along the sliding unit 220 down onto an empty space of the arrival unit 230 generated when the containers 5 are removed for a next process and may be seated on the arrival unit 230. Therefore, even when an additional container 5 is not injected into the container injector 200, processes may be continuously performed.

As a selective embodiment, a plurality of container injectors 200 may be arranged. In detail, four container injectors 200 may be provided. As an example, containers 5 having different capacities may be simultaneously injected into respective container injectors 200. As a detailed example, a container 5 having one capacity may be injected into two container injectors 200 selected from the four container injectors 200, and a container 5 having a different capacity from the container 5 injected into the two container injectors 200 selected above may be injected into the remaining two container injectors 200 that are not selected.

The robot arm 300 may move the container 5 seated on the arrival unit 220 to a location needed for each process. The robot arm 300 may be located on a central portion of the upper plate 80, may rotate in place, and may have a multi-joint structure. Also, the robot arm 300 may include a gripper 310 that may grip or place an object and thus may grip the container 5 and transport the container 5 to a desired location, move so that the container 5 heads in a desired direction, and put down the container 5 transported to the desired location so that the container 5 heads in the desired direction.

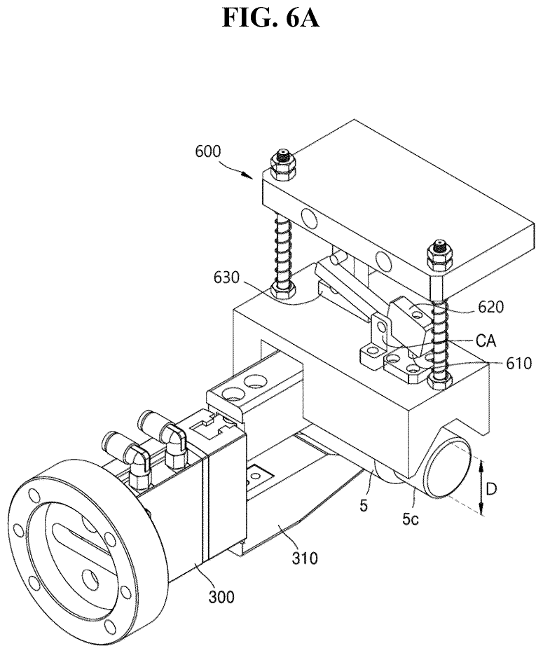

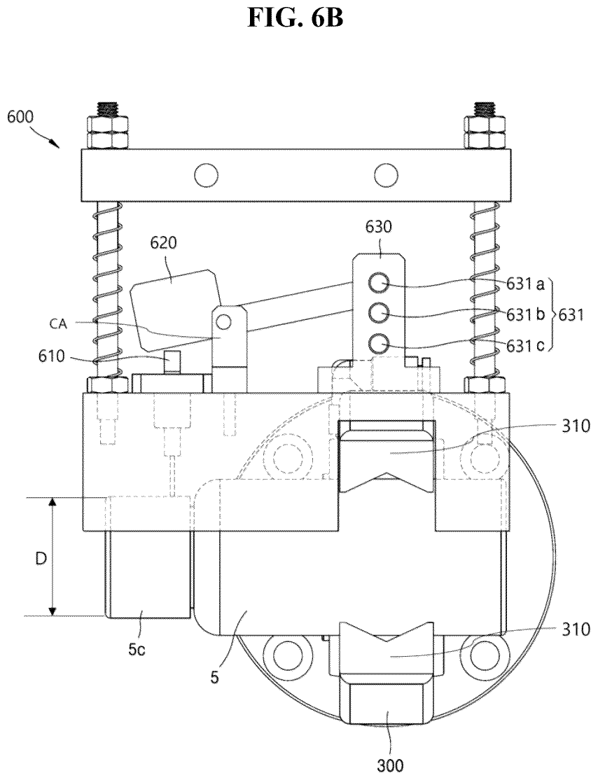

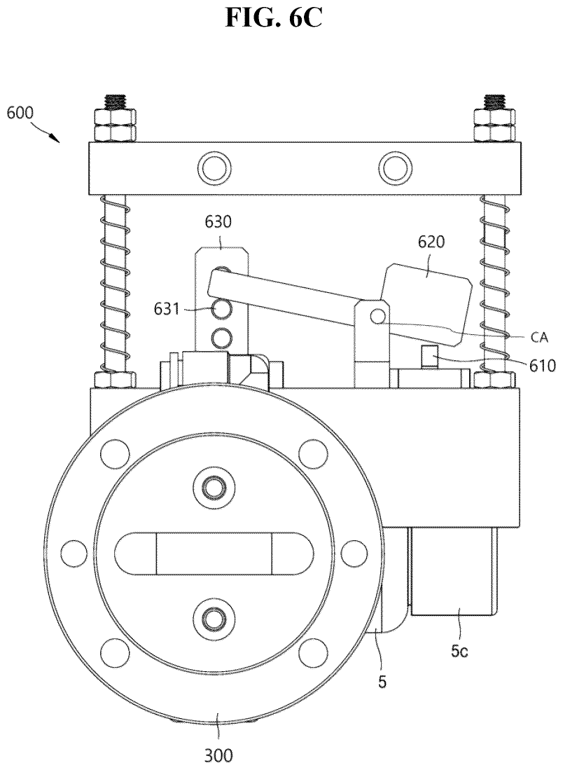

The stopper processing unit 700 may separate a stopper 5c of FIG. 6A covering an entrance of the container 5 from the container 5. As an example, a groove capable of fixing the stopper 5c of various sizes may be formed in a central portion of the stopper processing unit 700, and when the stopper 5c is inserted into the groove, the stopper 5c may be separated from the container 5 while a rotating apparatus mounted within the stopper processing unit 700 rotates in one direction. In addition, the separated stopper 5c may be fixed and mounted in the groove of the stopper processing unit 700 until the separated stopper 5c is re-coupled to the container 5.

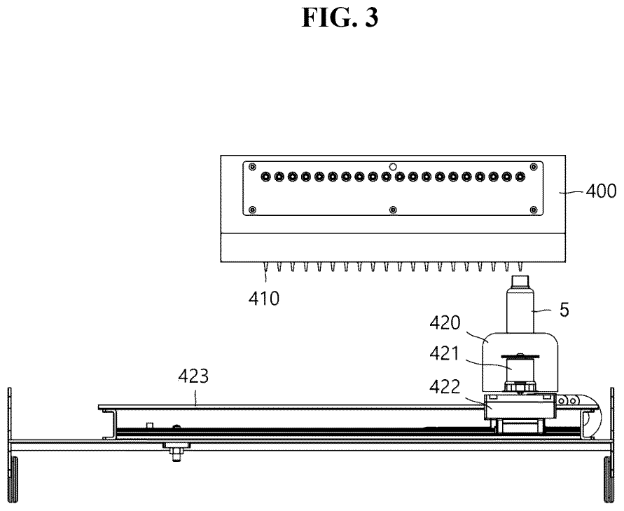

FIG. 3 is a plan view schematically illustrating a raw material injector of the apparatus of FIG. 1 for manufacturing customized cosmetic products.

Referring to FIG. 3, a raw material injector 400 may inject raw materials into a container 5 from which the stopper 5c of FIG. 6A is separated. As an example, the raw material injector 400 may include an injection nozzle 410 for accurately injecting the raw materials into the container 5. A plurality of injection nozzles 410 may be provided and may be arranged at regular intervals in a straight line along one surface of the raw material injector 400. In addition, the injection nozzles 410 may respectively discharge different raw materials.

As an example, to inject raw materials into the container 5, the robot arm 300 may grip the container 5 and move the container 5 back and forth along the injection nozzles 410 arranged in the straight line on one surface of the raw material injector 400. Here, the robot arm 300 may bring the container 5 closer to the injection nozzles 410 through vertical movement to prevent the raw materials from leaking out of the container 5.

As a selective embodiment, a container transport unit 420, which allows injection of the raw materials to be stably performed, may be further included. As an example, the container transport unit 420 may be connected to a moving unit 423 through a connection unit 422 and may move along the moving unit 423. The moving part 423 may be located on the same plane as the injection nozzles 410 arranged in the straight line along one surface of the raw material injector 400, and may be spaced apart from and formed parallel to the injection nozzles 410. Meanwhile, the container transport unit 420 may have a groove formed in a central portion thereof, in which the container 5 may be accommodated, and the groove may be formed to accurately match a location of the injection nozzles 410 when the container transport unit 420 moves along the moving unit 423. In addition, the container transport unit 420 may have a handle 421 formed on one surface thereof, so that the robot arm 300 may easily hold the handle 421 and transport the container transport unit 420. As an example, the container 5 may be accommodated in the groove formed in the central portion of the container transport unit 420, and the container transport unit 420 may move back and forth along the moving unit 423 formed parallel to the injection nozzles 410 so that the raw materials may be more stably injected into the container 5. Here, to prevent the raw materials from leaking out of the container 5, the robot arm 300 may move up and down to bring the container transport unit 420 closer to the injection nozzles 410.

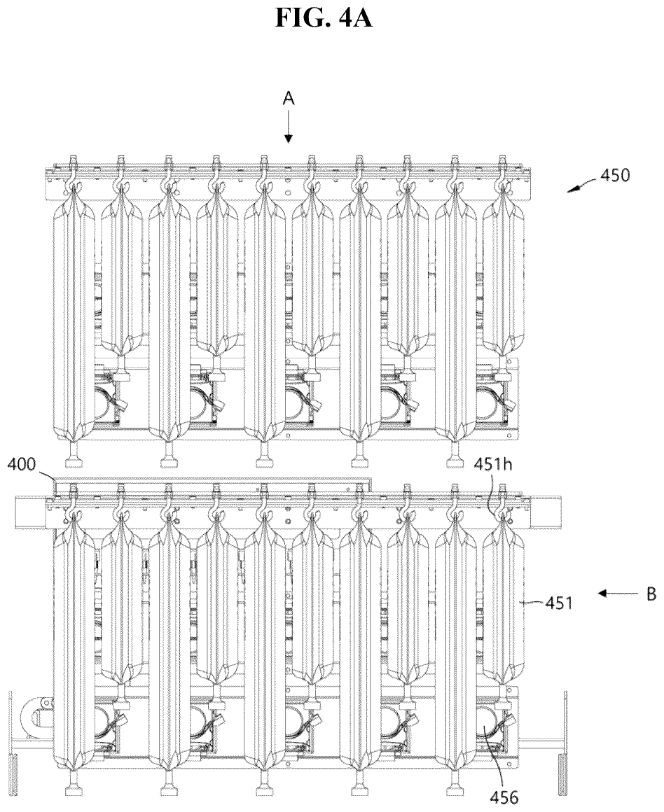

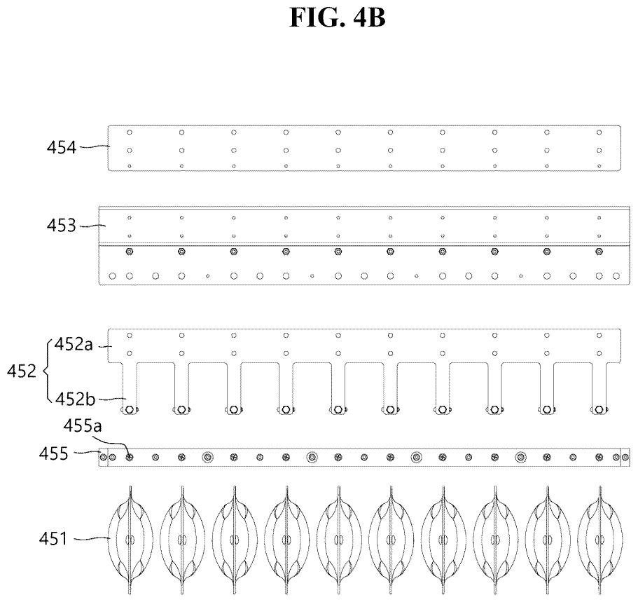

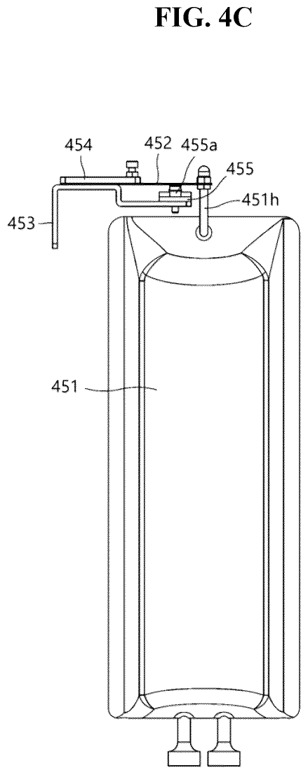

FIG. 4A is a plan view schematically illustrating a raw material supply unit of the apparatus of FIG. 1 for manufacturing customized cosmetic products, FIG. 4B is an exploded plan view of the raw material supply unit of FIG. 4A when viewed from a direction A, and FIG. 4C is a plan view of the raw material supply unit of FIG. 4A when viewed from a direction B.

Referring to FIGS. 4A to 4C, a raw material supply unit 450, which supplies raw materials to a raw material injector 400, may be further included. The raw material supply unit may include a mounting panel 452, a plurality of raw material packs 451, and a plurality of pumps 456, and the plurality of raw material packs 451 may be mounted on the mounting panel 452 by using hooks 451h of the mounting panel 452. Here, raw material packs may be mounted in two layers, and thus, various types of raw materials may be efficiently arranged in a limited space. In addition, the raw materials of the raw material packs 451 may be supplied to the raw material injector 400 by using the pumps 456 and raw material transport units (not shown), which are connected to both the raw material packs 451 and the raw material injector 400. As an example, the raw material transport units may include pipes, tubes, hoses, and the like, but are not limited thereto.

As a selective embodiment, the raw material supply unit 450 may include a detection panel 455, and the detection panel 455 may detect remaining amounts of the raw material packs 451. As an example, the detection panel 455 may be formed on one surface of the mounting panel 452. The mounting panel 452 and the detection panel 455 may be supported by a support panel 453, fixed to a fixing panel 454, and thus arranged between the support panel 453 and the fixing panel 454. Here, as an example, the mounting panel 452 may include a body portion 452a formed in a straight line and a plurality of protrusion portions 452b spaced apart from one another on one side of the body portion 452a and formed perpendicular to the body portion 452a. In addition, the mounting panel 452 may be formed of a material having elasticity and have a high elastic force. The material having the elasticity may be, for example, a polymer material, and may include, as a detailed example, pvc, acrylic, polypropylene, and polyethylene, but is not limited thereto.

Meanwhile, the raw material packs 451 may be mounted by enabling holes formed in at least portions of the raw material packs 451 to be hung on the hooks 451h arranged at ends of the protrusion portions 452b. Here, the protrusion portions 452b may be bent toward the raw material packs 451 according to the remaining amounts of the raw material packs 451. When the raw material packs 451 have large remaining amounts and thus are heavy, the protrusion portions 452b may be bent toward the raw material packs 451, and when the raw material packs 451 have small remaining amounts and thus are light, the protrusion portions 452b may return to original locations thereof by an elastic force.

Therefore, when the remaining amounts of the raw material packs 451 are large, the protrusion portions 452b may be bent toward the raw material packs 451 to be in contact with detection units 455a formed between the detection panel 455 and the mounting panel 452, and an electrical signal may be generated in the detection panel 455 by the detection units 455a. In contrast, when the remaining amounts of the raw material packs 451 are small, the protrusion portions 452b may be detached from the detection units 455a, and thus, the detection units 455a may no longer generate the electrical signal.

As a result, the raw material supply unit 450 may generate a replacement signal for a used raw material pack 451 by detecting the remaining amounts of the raw material packs 451.

Meanwhile, the stopper processing unit 700 may recouple the stopper 5c, which is fixed and mounted in the groove, to the entrance of the container 5 into which the raw materials are completely injected. As an example, when the container 5 is inserted into the stopper 5c, the rotating apparatus mounted within the stopper processing unit 700 may rotate in a different direction from the above-described one direction in which the stopper 5c is separated, and thus, the stopper 5c may be coupled to the container 5.

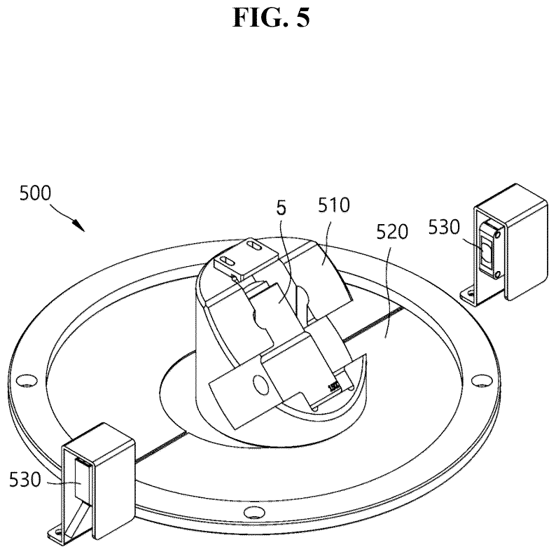

FIG. 5 is a perspective view schematically illustrating a stirring unit of the apparatus of FIG. 1 for manufacturing customized cosmetic products.

Referring to FIG. 5, a stirring unit 500 may mix raw materials injected into a container 5. As an example, the stirring unit 500 may include a seating unit 510 formed in a central portion of the stirring unit 500 and a rotator 520 that rotates in one direction so that the seating unit 510 rotates.

The seating unit 510 may be formed in the central portion of the stirring unit 500. As an example, the seating unit 510 may have a long groove formed in a central portion thereof to fit a size of the container 5. Here, the groove may be formed to form an acute angle with the upper plate 80 of FIG. 2, and thus, the container 5 into which the raw materials are injected may be safely mounted in the groove. In addition, a rotating unit (not shown), which is formed to rotate in contact with the container 5, may be mounted to allow the container 5 to rotate in place while being seated on the seating unit 510.

Meanwhile, the rotator 520 may rotate in one direction, and the seating unit 510 may be formed on the rotator 520. Accordingly, when the rotator 520 rotates in one direction, the seating unit 510 may also rotate in one direction together with the rotator 520.

As a result, the stirring unit 500 may perform biaxial stirring through a rotation in a first direction, which is performed by the rotator 520, and a rotation in a second direction, which is performed by the rotating unit (not shown) formed in the seating unit 510. When the stirring unit 500 performs biaxial stirring, the raw materials injected into the container 5 may be more uniformly mixed three-dimensionally.

The stirring unit 500 may perform biaxial stirring through the rotation in the first direction, which is performed by the rotator 520, and the rotation in the second direction, which is performed by the rotating unit (not shown) formed in the seating unit 510. When the stirring unit 500 performs biaxial stirring, the raw materials injected into the container 5 may be more uniformly mixed three-dimensionally.

Meanwhile, a second sensor unit 530 may include a pair of sensors that are arranged symmetrically with respect to the seating unit 510. As an example, the pair of sensors, which are symmetrically arranged with respect to the seating unit 510, may detect the container 5 seated on the seating unit 510. When the container 5 is detected as being seated on the seating unit 510, the stirring unit 500 may be operated.

A sensor may be, for example, a photoelectric sensor that detects light. The photoelectric sensor may include an emitting unit (not shown) that emits light and a light-receiving unit (not shown) that receives and detects light. Light emitted from each of the pair of sensors may pass through the container 5 seated on the seating unit 510 and be absorbed into the light-receiving unit (not shown) of the sensor opposite thereto. Here, an amount of light absorbed into the light-receiving unit (not shown) may be detected to identify whether or not the container is seated on the seating unit 510.

The discharge unit 800 may be formed in one region of the upper plate 80. The container 5 in which mixing is completed by the stirring unit 500 may be discharged through the discharge unit 800 to the outlet 50 formed in an outer surface.

Referring to FIG. 2, the apparatus 100 for manufacturing customized cosmetic products (hereinafter, referred to as the manufacturing apparatus) may include the housing 10, the transparent window 20 formed in the housing 10, the display unit 30, the barcode scanner 40, the outlet 50, the label output unit 60, the door 70, the plate 80, the container injector 200, the robot arm 300, the raw material injector 400, the stirring unit 500, the stopper processing unit 700, and a first discharge unit 800a and may further include an inspector 600 and a second discharge unit 800b.

The housing 10 may form the external appearance of the apparatus 100 for manufacturing customized cosmetic products. As an example, the housing 10 may have the hexahedral shape, and four pillars (not shown) may be located at vertices of the bottom of the hexahedral shape. The four pillars (not shown) may prevent the manufacturing apparatus 100 from tilting in any direction by adjusting the horizontality of the manufacturing apparatus 100. In addition, the housing 10 may include, on one surface thereof, the transparent window 20, the display unit 30, the barcode scanner 40, the outlet 50, and the label output unit 60. In addition, the housing 10 may include a plurality of doors 70 on remaining surfaces excluding one surface, the upper surface, and the lower surface thereof and may manage the interior of the manufacturing apparatus 100 through the doors 70.

Meanwhile, the interior of the housing 10 may be divided into the upper space S1 and the lower space S2 by the upper plate 80.

The upper plate 80 may form the bottom surface of the upper space S1 and may simultaneously form the upper surface of the lower space S2. Also, the lower plate (not shown) may be formed on the bottom surface of the lower space S2.

The power distribution box (not shown) for the electrical connections between the components of the manufacturing apparatus 100, the controller (not shown) for controlling the operation of the manufacturing apparatus 100, the container storage unit (not shown) for storing the container 5 of the cosmetic product, the raw material storage unit (not shown) for storing the cosmetic raw materials, the label issuer (not shown), and the like may be located on the lower plate (not shown).

Components for manufacturing a cosmetic product may be disposed on the upper plate 80. In detail, the container injector 200 into which the container 5 of the cosmetic product is injected, the robot arm 300 for moving the injected container 5 to a needed location for each process, the raw material injector 400 for injecting the raw materials into the container 5, the raw material supply unit 450 for supplying the raw materials to the raw material injector 400, the stirring unit 500 for mixing the raw materials injected into the container 5, the stopper processing unit 700, the first discharge unit 800a, and the second discharge unit 800b may be arranged, and the inspector 600 for inspecting the container 5 may be additionally arranged.

The container injector 200 may include the container injection inlet 210, the sliding unit 220, and the arrival unit 230. As an example, the container injection inlet 210 of the container injector 200 may be covered by the auxiliary door 75. The container injection inlet 210 may be covered by the auxiliary door 75, and thus, when the container 5 is injected, the entire door 70 does not need to be opened and foreign substances may be prevented from entering the container injector 200. Meanwhile, the container 5 may include two containers 5 having different capacities, and the two containers 5 having different capacities may have, for example, the same diameter and different lengths. The two containers 5 having different capacities may have the same diameter, may have different lengths, and thus may be equally injected into the same container injection inlet 210. When the container 5 is injected into the container injection inlet 210, the container 5 may move along the sliding unit 220 connecting the container injection inlet 210 to the arrival unit 230. Here, the container injection inlet 210 may be located higher than the arrival unit 230, and thus, the sliding unit 220 may have a slope, and the container 5 may be seated on the arrival unit 230 while sliding along the inclined sliding unit 220. In addition, a plurality of containers 5 may be loaded in the container injector 200 along the sliding unit 220. When the plurality of containers 5 are loaded along the sliding unit 220, the plurality of containers 5 may sequentially slide along the sliding unit 220 down onto the empty space of the arrival unit 230 generated when the containers 5 are removed for a next process and may be seated on the arrival unit 230. Therefore, even when an additional container 5 is not injected into the container injector 200, processes may be continuously performed.

As a selective embodiment, a plurality of container injectors 200 may be arranged. In detail, four container injectors 200 may be provided. As an example, containers 5 having different capacities may be simultaneously injected into respective container injectors 200. As a detailed example, a container 5 having one capacity may be injected into two container injectors 200 selected from the four container injectors 200, and a container 5 having a different capacity from the container 5 injected into the selected two container injectors 200 may be injected into the remaining two container injectors 200 that are not selected.

The robot arm 300 may move the container 5 seated on the arrival unit 220 to a location needed for each process. The robot arm 300 may be located on the central portion of the upper plate 80, may rotate in place, and may have the multi-joint structure. Also, the robot arm 300 may include the gripper 310 that may grip or place an object and thus may grip the container and transport the container 5 to a desired location, move so that the container 5 heads in a desired direction, and put down the container 5 transported to the desired location so that the container 5 heads in the desired direction.

FIG. 6A is a perspective view schematically illustrating an inspector of the apparatus of FIG. 2 for manufacturing customized cosmetic products, and FIGS. 6B and 6C are plan views schematically illustrating the inspector of FIG. 6A of the apparatus for manufacturing customized cosmetic products.

Referring to FIGS. 6A to 6C, an inspector 600 may distinguish between two containers having different capacities. By distinguishing between two containers having different capacities, the inspector 600 may distinguish a container incorrectly injected into another container injector 200 and prevent a defect from occurring during manufacturing. As an example, to distinguish between two containers having different capacities, the inspector 600 may include a lever 620 that seesaws around a central axis CA, a pin 610 arranged on one side of the lever 620, and a first sensor unit 630 arranged on the other side of the lever 620.

The pin 610 may be arranged on the one side of the lever 620 to be perpendicular to the lever 620 and may be move in a vertical direction. As an example, the pin 610 may be raised by a container 5 moved to the inspector 600 by a robot arm 300. In detail, the container 5 moved to the inspector 600 by the robot arm 300 may be placed in a direction perpendicular to the pin 610 by the robot arm 300 and may be lifted toward the pin 610 by the robot arm 300. Here, a portion of a stopper 5c of the container 5 may contact the pin 610, and when the stopper 5c contacts the pin 610, the pin 610 may be raised.

The lever 620 may be arranged perpendicular to the pin 610, may be fixed to the central axis CA, and perform seesaw movement. In addition, the one side of the lever 620 may be heavier than the other side of the lever 620, and thus, the one side of the lever 620 may be inclined toward the pin 610 to always contact the pin 610. Therefore, when the pin 610 is raised by the stopper 5c, the one side of the lever 620 may also be raised together with the pin 610, and when the central axis CA of the lever 620 is fixed, the other side of the lever 620 may be lowered.

The first sensor unit 630 may be arranged on the other side of the lever 620 to be perpendicular to the lever 620 and may include a plurality of sensors 631. In detail, the first sensor unit 630 may include three sensors 631. As an example, when any one of the three sensors 631 is covered with the other side of the lever 620, the first sensor unit 630 may distinguish each of the containers 5 having different capacities.

Meanwhile, a method by which the inspector 600 detects the containers 5 having different capacities may be based on diameters D of the stoppers 5c of the containers 5. The stoppers 5c may have different diameters D according to capacities of the containers 5, when the diameters D of the stoppers 5c are different, a rise width of the pin 610 lifted by the stoppers 5c may vary, and when the rise width of the pin 610 varies, a rise width of the one side of the lever 620 may vary and a falling width of the other side of the lever 620 may also vary. Accordingly, according to the diameter D of the stopper 5c, the rise width of the pin 610 may be determined, the rise width of the one side of the lever 620 may also be determined, and as a result, the falling width of the other side of the lever 620 may be also determined. In other words, a location of the other side of the lever 620 may be determined by the diameter D of the stopper 5c.

Therefore, when the location of the other side of the lever 620 is determined by the diameter D of the stopper 5c, any one of the three sensors 631 may be covered according to the location of the other side of the lever 620. Here, the inspector 600 may distinguish between the containers 5 having different capacities by the sensor 631 that is covered.

As an example, when the other side of the lever 620 covers a first sensor 631a of the first sensor unit 630, the container 5 may be removed from the inspector 600 while the stopper 5c of the container 5 may not push up the pin 610. When the other side of the lever 620 covers a second sensor 631b or a third sensor 631c, the container 5 may be loaded on the inspector 600 while the stopper 5c of the container 5 pushes up the pin 610. Here, when the other side of the lever 620 covers the second sensor 631b, the stopper 5c may push up the pin 610 slightly higher than when covering the third sensor 631c, and thus, the diameter D of the stopper 5c may be smaller than when the other side of the lever 620 covers the third sensor 631c. In contrast, when the other side of the lever 620 covers the third sensor 631c, the stopper 5c may push up the pin 610 more than when covering the second sensor 631b, and thus, the diameter D of the stopper 5c may be greater than when the other side of the lever 620 covers the second sensor 632b. Therefore, the sensor 631, which is covered by the other side of the lever 620, may be determined by the diameter D of the stopper 5c, and as a result, the inspector 600 may distinguish between the containers 5 having different capacities.

Meanwhile, the container 5, which is completely distinguished, may proceed to a next process or be accommodated in the second discharge unit 800b and thus discharged into the lower space S2, according to the result of distinguishment.

The second discharge unit 800b may be formed in one region of the upper plate 80 to be connected to the lower space S2. When the container 5 distinguished by the inspector 600 is a container 5 inappropriate for a process rather than a container 5 appropriate for the process, the container 5 may be discharged into the lower space S2 through the second discharge unit 800b.

The stopper processing unit 700 may separate, from the container 5, the stopper 5c covering the entrance of the container 5 that is completely distinguished. As an example, the groove capable of fixing the stopper 5c of various sizes may be formed in the central portion of the stopper processing unit 700, and when the stopper 5c is inserted into the groove, the stopper 5c may be separated from the container 5 while the rotating apparatus mounted within the stopper processing unit 700 rotates in one direction. In addition, the separated stopper 5c may be fixed and mounted in the groove of the stopper processing unit 700 until the separated stopper 5c is recoupled to the container 5.

The raw material injector 400 may inject the raw materials into the container 5 from which the stopper 5c is separated. As an example, the raw material injector 400 may include the injection nozzles 410 for accurately injecting the raw materials into the container 5. A plurality of injection nozzles 410 may be provided and may be arranged at regular intervals in a straight line along one surface of the raw material injector 400. In addition, the injection nozzles 410 may respectively discharge different raw materials.

As an example, to inject the raw materials into the container 5, the robot arm 300 may grip the container 5 and move the container 5 back and forth along the injection nozzles 410 arranged in the straight line on one surface of the raw material injector 400. Here, the robot arm 300 may bring the container 5 closer to the injection nozzles 410 through vertical movement to prevent the raw materials from leaking out of the container 5.

As a selective embodiment, the container transport unit 420, which allows the injection of the raw materials to be stably performed, may be further included. As an example, the container transport unit 420 may be connected to the moving unit 423 through the connection unit 422 and may move along the moving unit 423. The moving unit 423 may be located on the same plane as the injection nozzles 410 arranged in the straight line along one surface of the raw material injector 400, and may be spaced apart from and formed parallel to the injection nozzles 410. Meanwhile, the container transport unit 420 may have the groove formed in the central portion thereof, in which the container 5 may be accommodated, and the groove may be formed to accurately match the location of the injection nozzle 410 when the container transport unit 420 moves along the moving unit 423. In addition, the container transport unit 420 may have the handle 421 formed on one surface thereof, so that the robot arm 300 may easily hold the handle 421 and transport the container transport unit 420. As an example, the container 5 may be accommodated in the groove formed in the central portion of the container transport unit 420, and the container transport unit 420 may move back and forth along the moving unit 423 formed parallel to the injection nozzles 410 so that the raw materials may be more stably injected into the container 5. Here, to prevent the raw materials from leaking out of the container 5, the robot arm 300 may move up and down to bring the container transport unit 420 closer to the injection nozzles 410.

The raw material supply unit 450 may include the mounting panel 452, the plurality of raw material packs 451, and the plurality of pumps 456, and the plurality of raw material packs 451 may be mounted on the mounting panel 452 by using the hooks 451h of the mounting panel 452. Here, raw material packs may be mounted in two layers, and thus, various types of raw materials may be efficiently arranged in a limited space. In addition, the raw materials of the raw material packs 451 may be supplied to the raw material injector 400 by using the pumps 456 and the raw material transport units (not shown), which are connected to both the raw material packs 451 and the raw material injector 400. As an example, the raw material transport units may include the pipes, the tubes, the hoses, and the like, but are not limited thereto.

As a selective embodiment, the raw material supply unit 450 may include the detection panel 455, and the detection panel 455 may detect remaining amounts of the raw material packs 451. As an example, the mounting panel 452 may include the body portion 452a formed in the straight line and the plurality of protrusion portions 452b spaced apart from one another on one side of the body portion 452a and formed perpendicular to the body portion 452a. In addition, the mounting panel 452 may be formed of a material having elasticity and have a high elastic force.

Meanwhile, the raw material packs 451 may be mounted by enabling the holes formed in at least portions of the raw material packs 451 to be hung on the hooks 451h arranged at the ends of the protrusion portions 452b. When the remaining amounts of the raw material packs 451 are large, the protrusion portions 452b may be bent toward the raw material packs 451 to be in contact with the detection units 455a formed between the detection panel 455 and the mounting panel 452, and an electrical signal may be generated in the detection panel 455 by the detection units 455a. In contrast, when the remaining amounts of the raw material packs 451 are small, the protrusion portions 452b may be detached from the detection units 455a, and thus, the detection panel 455 may no longer generate the electrical signal.

As a result, the raw material supply unit 450 may generate a replacement signal for a used raw material pack 451 by detecting the remaining amounts of the raw material packs 451.

Meanwhile, the stopper processing unit 700 may recouple the stopper 5c, which is fixed and mounted in the groove, to the entrance of the container 5 into which the raw materials are completely injected. As an example, when the container 5 is inserted into the stopper 5c, the rotating apparatus mounted within the stopper processing unit 700 may rotate in a different direction from the above-described one direction in which the stopper 5c is separated, and thus, the stopper 5c may be coupled to the container 5.

The stirring unit 500 may mix the raw materials injected into the container 5. As an example, the stirring unit 500 may include the seating unit 510 formed in the central portion of the stirring unit 500 and the rotator 520 that rotates in one direction so that the seating unit 510 rotates.

The seating unit 510 may be formed in the central portion of the stirring unit 500. As an example, the seating unit 510 may have the long groove formed in the central portion thereof to fit the size of the container 5. Here, the groove may formed to form an acute angle with the upper plate 80 of FIG. 2, and thus, the container 5 into which the raw materials are injected may be safely mounted in the groove. In addition, the rotating unit (not shown), which is formed to rotate in contact with the container 5, may be mounted to allow the container 5 to rotate in place while being seated on the seating unit 510.

Meanwhile, the rotator 520 may rotate in one direction, and the seating unit 510 may be formed on the rotator 520. Accordingly, when the rotator 520 rotates in one direction, the seating unit 510 may also rotate in one direction together with the rotator 520.

As a result, the stirring unit 500 may perform biaxial stirring through the rotation in the first direction, which is performed by the rotator 520, and the rotation in the second direction, which is performed by the rotating unit (not shown) formed in the seating unit 510. When the stirring unit 500 performs biaxial stirring, the raw materials injected into the container may be more uniformly mixed three-dimensionally.

Meanwhile, the second sensor unit 530 may include a pair of sensors that are arranged symmetrically with respect to the seating unit 510. As an example, the pair of sensors symmetrically arranged with respect to the seating unit 510 may detect the container 5 seated on the seating unit 510. When the container 5 is detected as being seated on the seating unit 510, the stirring unit 500 may be operated.

The sensor may be, for example, the photoelectric sensor that detects light. The photoelectric sensor may include the emitting unit (not shown) that emits light and the light-receiving unit (not shown) that receives and detects light. Light emitted from each of the pair of sensors may pass through the container 5 seated on the seating unit 510 and be absorbed into the light-receiving unit (not shown) of the sensor opposite thereto. Here, an amount of light absorbed into the light-receiving unit (not shown) may be detected to identify whether or not the container 5 is seated on the seating unit 510.

The first discharge unit 800a may be formed in one region of the upper plate 80. The container 5 in which mixing is completed by the stirring unit 500 may be discharged through the first discharge unit 800a to the outlet 50 formed in the outer surface.

As a result, an apparatus for manufacturing customized cosmetic products may detect a remaining amount of a raw material pack to prevent a defect in a cosmetic product, which may occur when raw materials are used up and thus raw materials are missing during manufacturing of the cosmetic product.

According to embodiments, there may be provided an apparatus for manufacturing customized cosmetic products, which may detect a remaining amount of a raw material pack to prevent a defect in a cosmetic product, which may occur when raw materials are used up and thus raw materials are missing during manufacturing of the cosmetic product.

Although the embodiments have been illustrated and described above, the disclosure is not limited to the particular embodiments described above, various modifications may be implemented by one of ordinary skill in the art without departing from the spirit of the disclosure claimed by claims, and these modifications should not be individually understood from the spirit or prospect of the disclosure.

Therefore, the spirit of the disclosure should not be limited to the above-described embodiments, and not only claims described below but also all scopes equivalent to or equivalently changed from the claims fall within the scope of the spirit of the disclosure.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. An apparatus for manufacturing customized cosmetic products, the apparatus comprising:
a container injector configured to inject a container;
a robot arm configured to move the injected container to a location needed for each process;
a raw material injector configured to inject raw materials for a product into the container;
a raw material supply unit configured to supply the raw materials to the raw material injector; and
a stirring unit configured to mix the raw materials injected into the container, wherein the raw material supply unit comprises: a mounting panel on which a raw material pack is mounted;
and a detection panel arranged on one surface of the mounting panel, and the mounting panel has elasticity and is in contact with or detached from the detection panel according to a remaining amount of the raw material pack.

2. The apparatus of claim 1, wherein the detection panel comprises a plurality of detection units formed to be spaced apart from one another along one surface of the detection panel, a signal is generated by contact or separation between the mounting panel and the detection units, and the raw material pack, which needs to be replaced, is determined according to the signal.

3. The apparatus of claim 1, wherein the mounting panel comprises a body portion formed in a straight line and a plurality of protrusion portions spaced apart from one another on one side of the body portion and formed perpendicular to the body portion, raw material packs are mounted on the protrusion portions, and the protrusion portions are in contact with or detached from the detection panel according to remaining amounts of the raw material packs.

4. The apparatus of claim 1, further comprising:
an inspector configured to distinguish between containers having different capacities, wherein the inspector comprises: a lever seesawing around a central axis; a pin arranged on one side of the lever and movable in a vertical direction; and a first sensor unit arranged on another side of the lever, and when the pin rises, the one side of the lever is raised by the pin, the other side of the lever is lowered around the central axis, and the first sensor unit detects a location of the other side of the lever.

5. The apparatus of claim 4, wherein the first sensor unit comprises a plurality of sensors arranged in the vertical direction, and the sensors detect the location of the other side of the lever, and the containers having different capacities are distinguished by the location of the other side of the lever.

6. The apparatus of claim 4, wherein the pin is raised by the container, and the container is arranged perpendicular to the pin by the robot arm and is pushed up by the robot arm.

7. The apparatus of claim 1, wherein the stirring unit comprises a seating unit on which the container is seated, the seating unit rotates in a first direction, and the container rotates in a second direction within the seating unit.

8. The apparatus of claim 1, further comprising:
a second sensor unit comprising a pair of sensors arranged symmetrically around the stirring unit.

9. The apparatus of claim 8, wherein the pair of sensors comprise photo sensors, and the container is located between the pair of sensors.

10. The apparatus of claim 1, further comprising:
a first discharge unit and a second discharge unit configured to discharge the container, wherein the first discharge unit accommodates the container in which the raw materials are mixed by the stirring unit, and the second discharge unit accommodates the container determined to be inappropriate by the inspector.

11. The apparatus of claim 5, further comprising:
a first discharge unit and a second discharge unit configured to discharge the container, wherein the first discharge unit accommodates the container in which the raw materials are mixed by the stirring unit, and the second discharge unit accommodates the container determined to be inappropriate by the inspector.
